(19) United States
(12) Patent Application Publication
(10) Pub. No.: US 2014/0322761 A1
(43) Pub. Date: Oct. 30, 2014

(54) METHOD OF PREPARING SAMPLE FOR NUCLEIC ACID AMPLIFICATION REACTION, NUCLEIC ACID AMPLIFICATION METHOD, AND REAGENT AND MICROCHIP FOR SOLID PHASE NUCLEIC ACID AMPLIFICATION REACTION

(71) Applicant: Sony Corporation, Tokyo (JP)

(72) Inventors: Tomohiko Nakamura, Tokyo (JP); Kenzo Machida, Kanagawa (JP)

(73) Assignee: SONY CORPORATION, Tokyo (JP)

(21) Appl. No.: 14/190,865

(22) Filed: Feb. 26, 2014

(30) Foreign Application Priority Data
Mar. 29, 2013 (JP) 2013-075428

(51) Int. Cl. C12Q 1/68

(52) U.S. Cl. CPC C12Q 1/6806
USPC 435/91.21; 435/91.2; 435/194; 435/289.1

(57) ABSTRACT

Provided is a method of preparing a sample for nucleic acid amplification reaction, including: a procedure of dissolving a solid phase reagent at least containing DNA polymerase, cyclodextrin, and a binder, in a liquid containing a nucleic acid.

### CROSS REFERENCE TO RELATED APPLICATIONS

This application claims the benefit of Japanese Priority Patent Application JP 2013-075428 filed Mar. 29, 2013, the entire contents of which are incorporated herein by reference.

### BACKGROUND

The present technology relates to a method of preparing a sample for nucleic acid amplification reaction, a nucleic acid amplification method, and a reagent and a microchip for solid phase nucleic acid amplification reaction. More specifically, the present technology relates to a solid phase reagent used for preparing the sample for nucleic acid amplification reaction, or the like.

The nucleic acid amplification reaction is a reaction that newly synthesizes a nucleic acid complementary to a nucleic acid used as a template. A plurality of reagents such as an oligonucleotide called a primer or oxygen in addition to the nucleic acid used as a template are necessary in order to perform the nucleic acid amplification reaction. A sample for the nucleic acid amplification reaction is prepared by mixing the reagents and the nucleic acid used as a template in order to perform the nucleic acid amplification reaction.

In the related art, the nucleic acid amplification reaction has been performed by inputting the above-described reagents and the template nucleic acid into a microchip or the like and mixing them together, and then by transferring the obtained sample for the nucleic acid amplification reaction into a suitable container. In addition, in recent years, a reagent that is stored in a state such as a microchip where a plurality of reagents necessary for the nucleic acid amplification reaction are mixed in advance has been developed. In some cases, such a mixed reagent is accommodated in a base material used for the nucleic acid amplification reaction.

Japanese Unexamined Patent Application Publication No. 2011-160728 discloses “a microchip for a nucleic acid amplification reaction including an entrance through which a liquid enters from the outside, a plurality of wells configured to function as reaction sites of nucleic acid amplification reaction, and flow channels through which the liquid entered from the entrance is fed into each of the wells, in which a plurality of reagents necessary for the reaction are laminated and anchored in a prescribed order in each well”.

### SUMMARY

In the microchip disclosed in the above-described Japanese Unexamined Patent Application Publication No. 2011-160728, it is possible to make the sample including the template nucleic acid enter the microchip and to simply perform the nucleic acid amplification reaction by anchoring the plurality of reagents necessary for the reaction in the wells. In addition, a further improvement in preparing the samples has been expected in order to more simply perform the nucleic acid amplification reaction.

In the present technology, it is desirable to provide a method of preparing a sample for nucleic acid amplification reaction capable of simply and accurately performing nucleic acid amplification reaction.

According to an embodiment of the present technology, there is provided a method of preparing a sample for nucleic acid amplification reaction including a procedure of dissolving a solid phase reagent at least containing DNA polymerase, cyclodextrin, and a binder, in a liquid containing a nucleic acid.

In the embodiment, the method may include a procedure of diluting the liquid with a solution containing an ionic surfactant prior to the procedure of the dissolving of the solid phase reagent.

In the embodiment, the ionic surfactant may be an anionic surfactant and the anionic surfactant may be sodium dodecyl sulfate.

In the embodiment, the concentration of the cyclodextrin may be higher than or equal to 8 times the concentration of the sodium dodecyl sulfate.

In the embodiment, the method may include a procedure of performing sonication of the diluted solution of the liquid prior to the procedure of the dissolving of the solid phase reagent and include a procedure of heating the diluted solution of the liquid prior to the procedure of the dissolving of the solid phase reagent.

According to another embodiment of the present technology, there is provided a nucleic acid amplification method including a procedure of dissolving a solid phase reagent at least containing DNA polymerase, cyclodextrin, and a binder, in a liquid containing a nucleic acid; and a procedure of amplifying the nucleic acid.

In the embodiment, amplification of the nucleic acid may be isothermally performed. In addition, the nucleic acid may be the ribonucleic acid and the nucleic acid amplification method may further include a procedure of performing reverse transcription reaction using a ribonucleic acid as a template prior to the procedure of the amplifying of the nucleic acid.

According to still another embodiment of the present technology, there is provided a reagent for solid phase nucleic acid amplification reaction including at least a DNA polymerase, cyclodextrin, and a binder.

In the embodiment, the cyclodextrin may include a hydroxypropyl group.

In the embodiment, the reagent for solid phase nucleic acid amplification reaction may be mixed in a liquid containing a template nucleic acid chain and an ionic surfactant.

In the embodiment, the concentration of the cyclodextrin may be higher than or equal to 8 times the concentration of the ionic surfactant.

In the embodiment, the reagent for solid phase nucleic acid amplification reaction may further include ribonuclease H.

According to still another embodiment of the present technology, there is provided a microchip including a reagent for solid phase nucleic acid amplification reaction at least containing DNA polymerase, cyclodextrin, and a binder.

In the embodiment, the reagent for solid phase nucleic acid amplification reaction may be provided in each of a plurality of reaction sites of nucleic acid amplification reaction installed in the microchip, and the reaction sites may communicate with an entrance through which a liquid enters the microchip, through flow channels.

According to the embodiments of the present technology, there is provided a method of preparing a sample for nucleic acid amplification reaction capable of simply and accurately performing nucleic acid amplification reaction.

### BRIEF DESCRIPTION OF THE DRAWINGS

FIG. 1 is a flowchart of a method of preparing a sample for nucleic acid amplification reaction according to an embodiment of the present technology;

FIGS. 2A and 2B are schematic views that show an example of a configuration of a microchip according to an embodiment of the present technology. FIG. 2A is a top view and FIG. 2B is a cross-sectional view taken along line IIB-IIB of an arrow of FIG. 2A;

FIG. 3 is a graph substitute for a drawing that illustrates a relationship between a concentration of SDS and an activity of RNase A (Example 3);

FIG. 4 is a graph substitute for a drawing that illustrates a relationship between a concentration of SDS and an activity of RNase A contained in plasma (Example 4);

FIG. 5 is a graph substitute for a drawing that illustrates a relationship between a concentration of SDS and an activity of RNase A contained in plasma (Example 4);

FIG. 6 is a graph substitute for a drawing that illustrates a relationship between a concentration of SDS and a Tt value in nucleic acid amplification reaction where a bacterial genome is used as a template nucleic acid (Example 5);

FIG. 7 is a graph substitute for a drawing that illustrates a relationship between a concentration of SDS and a concentration of cyclodextrin in a nucleic acid amplification reaction (Example 6); and

FIG. 8 is a graph substitute for a drawing that illustrates a relationship between a concentration of RNase H and a Tt value in nucleic acid amplification reaction accompanied with reverse transcription reaction (Example 10).

### DETAILED DESCRIPTION OF EMBODIMENTS

Hereinafter, preferable embodiments of the present technology are described. The embodiments described below only illustrate typical embodiments of the present technology and they do not limit the scope of the present technology.

1. Reagent for Solid Phase Nucleic Acid Amplification Reaction According to Embodiment of Present technology

A reagent for solid phase nucleic acid amplification reaction according to an embodiment of the present technology (hereinafter, also simply referred to as “solid phase reagent”) contains at least DNA polymerase, cyclodextrin, and a binder. Each component included in the solid phase reagent will be described in the aforementioned order.

### (1) DNA Polymerase

The DNA polymerase contained in the solid phase reagent is a component for synthesizing a nucleic acid chain complementary to a template nucleic acid in nucleic acid amplification reaction. The DNA polymerase can be appropriately selected based on an arbitrary nucleic acid amplification method. Examples of the DNA polymerase include Taq DNA polymerase, Tth DNA polymerase, KOD DNA polymerase, and Pfu DNA polymerase. In addition, chain substitution type DNA polymerase may be included therein.

### (2) Cyclodextrin

The cyclodextrin contained in the solid phase reagent is a component for suppressing deterioration of activity of an enzyme such as the DNA polymerase contained in the solid phase reagent (refer to Example 1). The solid phase reagent is prepared such that a reagent is made into a predetermined component and the predetermined component is subsequently dried or freeze-dried. There is a concern that an enzyme contained in the solid phase reagent is deactivated depending on such a preparation process or a dried state after the preparation process. In the solid phase reagent according to an embodiment of the present technology, it is possible to suppress the deterioration of the activity of the enzyme using the reagent containing the cyclodextrin (refer to Example 1).

In addition, the cyclodextrin also has an effect of suppressing inhibition of nucleic acid amplification reaction of an ionic surfactant contained in a liquid containing a nucleic acid to be described (refer to Example 2). In a case where the liquid containing the nucleic acid contains the ionic surfactant, it is preferable that the concentration of the cyclodextrin be higher than or equal to 8 times the concentration of the ionic surfactant in the solid phase reagent in order to obtain the suppressing effect (refer to Example 6).

Examples of the cyclodextrin include α-cyclodextrin (the number of glucoses: 6), β-cyclodextrin (the number of glucoses: 7), γ-cyclodextrin (the number of glucoses: 8), and derivatives thereof. The derivatives of the cyclodextrin are molecules to which a part of hydroxyl group is substituted to an OR group. Examples of R include a hydrocarbon group such as a methyl group and a ethyl group, and a hydroxyalkyl group such as a hydroxyethyl group and a hydroxyprophyl group.

In the solid phase reagent according to an embodiment of the present technology, it is preferable that the cyclodextrin have the cyclodextrin group, for example, hydroxyprophyl-β-cyclodextrin (HPβCD). Since HPβCD has higher water solubility compared to the β-cyclodextrin, it is easy to add a sufficient amount of cyclodextrin to the solid phase reagent in order to obtain an effect with respect to the ionic surfactant to be described.

### (3) Binder

The binder contained in the solid phase reagent is a component for enhancing stability of the shape of the solid phase reagent. In particular, in a case there cyclodextrin such as the above-described HPβCD having high hygroscopicity is included in the solid phase reagent, it is difficult to maintain the shape of the solid phase reagent. Therefore, the shape of the solid phase reagent according to an embodiment of the present technology can be maintained by adding the binder.

As the binder, any component may be used as long as the component does not inhibit the nucleic acid amplification reaction. Examples of the binder include carbohydrates such as sucrose, dextran, trehalose, and FICOLL; proteins or peptides such as collagen peptides, gelatin, and BSA; and polymeric compounds such as polyethylene glycol (PEG) and polyvinyl pyrrolidone. The solid phase reagent containing the binder can be prepared such that a binder dissolution liquid containing the above-described components is mixed with a liquid-type or gel-type reagent containing the DNA polymerase or the like and the mixture is subsequently dried or freeze-dried.

### (4) Ribonuclease H

The solid phase reagent according to an embodiment of the present technology may contain ribonuclease H (RNase H). The RNase H is an enzyme that specifically hydrolyzes an RNA chain of an RNA/DNA hybrid chain. The solid phase reagent containing the ribonuclease H (RNase H) is suitable for nucleic acid amplification reaction accompanied with reverse transcription reaction and is suitable for an isothermal nucleic acid amplification method. The nucleic acid amplification reaction accompanied with the reverse transcription reaction is a technique that can promptly detect RNA by continuously performing the reverse transcription reaction and the nucleic acid amplification reaction compared to a case where the reactions are separately performed in two steps.

In the reverse transcription reaction, DNA is synthesized using RNA as a template, but the synthesized DNA is in a state where the DNA is hybridized with the RNA used as a template. In the isothermal nucleic acid amplification method, since there is no process where a reaction temperature is raised, a nucleic acid in a state of an RNA/DNA hybrid chain is denatured thereby making a single chain, there is a concern that efficiency of the nucleic acid amplification reaction using the synthesized DNA as a template is deteriorated. By including RNase H in the solid phase reagent, DNA and RNA hybridized with the DNA are decomposed to make the DNA be a single chain, thereby the nucleic acid amplification reaction can be more efficiently performed. There is also reverse transcriptase including an RNase H activity. However, it is possible to more efficiently perform the nucleic acid amplification reaction if a reagent contains RNase H compared to a case of using only the RNase H activity of the reverse transcriptase (refer to Example 10).

The solid phase reagent according to an embodiment of the present technology may contain a component necessary for the nucleic acid amplification reaction in addition to the components described above. Examples of the components contained in the solid phase reagent include dNTP or a primer, and a component contained in a buffer solution for stabilizing the nucleic acid amplification reaction. In order to suppress the decomposition while completing the reverse transcription reaction of the RNA chain, an inhibitor against RNase A may be included in the solid phase reagent. In addition, the activity of the above-described RNase H is not interfered from the RNase A inhibitor. Therefore, in a case of using a solid phase sample for the nucleic acid amplification reaction accompanied with the reverse transcription reaction, it is preferable that the solid phase reagent contain the RNase H and the RNase A inhibitor (refer to Example 11).

### 2. Method of Preparing Sample for Nucleic Acid Amplification Reaction According to Embodiment of Present Technology

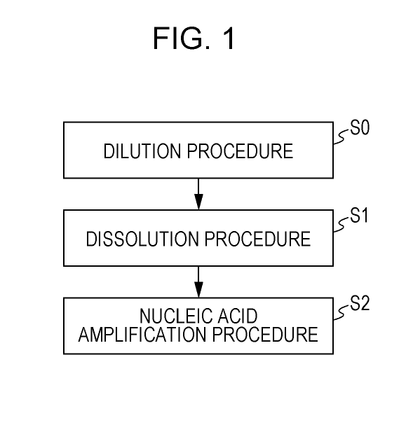

The reagent for solid phase nucleic acid amplification reaction described above can be suitably used for a method of preparing a sample for nucleic acid amplification reaction according to an embodiment of the present technology (hereinafter, also simply referred to as “method of preparing a sample”). FIG. 1 is a flowchart of a method of preparing a sample for nucleic acid amplification reaction according to an embodiment of the present technology. The method of preparing the sample includes a procedure of dissolving a solid phase reagent (reagent for solid phase nucleic acid amplification reaction) in a liquid containing a nucleic acid (dissolving procedure S1). In addition, the method of preparing the sample may include a procedure that dilutes the liquid with a solution containing an ionic surfactant (dilution procedure S0) prior to the procedure of the dissolving of the solid phase reagent (dissolution procedure S1). Furthermore, the nucleic acid amplification method of an embodiment of the present technology may contain a procedure of amplifying a nucleic acid (amplification procedure S2) in addition to the procedures. Each procedure shown in FIG. 1 is described.

### (1) Dissolution Procedure

In the dilution procedure S1, the above-described solid phase reagent is dissolved in a liquid containing a nucleic acid used as a template nucleic acid in the nucleic acid amplification reaction. In the method of preparing a sample according to an embodiment of the present technology, the nucleic acid is a nucleic acid derived from animals, plants, fungi, bacteria, viruses, or the like. The nucleic acid may be any of a single chain nucleic acid and a double chain nucleic acid and may be any of DNA and RNA. In addition, molecular weight of the nucleic acid is also not particularly limited. The nucleic acid contained in the sample may be in a state of being surrounded by a cell membrane or in a state of existing in a particle such as a bacterial genome existed in a cell of a bacterium, without being directly dispersed in the sample.

In the method of preparing a sample according to an embodiment of the present technology, any liquid containing the nucleic acid may be used as long as the liquid contains the above-described nucleic acid. However, it is preferable that the nucleic acid be contained in a solvent in which the nucleic acid in the liquid is rarely decomposed and that does not have a component of inhibiting the nucleic acid amplification reaction. Examples of the solvent include various buffer solutions such as a Tris buffer solution, and water. The liquid containing the nucleic acid may be an organism-derived sample or a diluted solution of the same. Examples of the organism-derived sample include whole blood, plasma, serum, cerebrospinal fluid, urine, semen, swabs (that have the swabbed liquid of the nose and throat, nasal mucus, sputum, or the like), saliva, or the like. In addition, the liquid containing the nucleic acid may be a gel-type liquid.

In the method of preparing a sample according to an embodiment of the present technology, since the solid phase reagent contains DNA polymerase, it is possible to easily prepare a sample for nucleic acid amplification reaction by dissolving the solid phase reagent in the liquid containing the nucleic acid. Furthermore, since the solid phase reagent contains cyclodextrin, the deterioration of the activity of the DNA polymerase contained in the solid phase reagent is suppressed. Accordingly, in the method of preparing a sample using the solid phase reagent according to an embodiment of the present technology, it is possible to simply prepare a sample and accurately implement the nucleic acid amplification reaction.

### (2) Dilution Procedure

In the dilution procedure S0 shown in FIG. 1, the liquid containing the nucleic acid described above is diluted with a solution containing an ionic surfactant. Examples of the ionic surfactant include cationic surfactant such as hexadecyltrimethylammonium bromide and myristyltrimethylammonium bromide, and anionic surfactant such as sodium dodecyl sulfate (SDS) and sodium deoxycholate. As the ionic surfactant, the anionic surfactant is preferable and the SDS is more preferable.

It is possible to inhibit an activity of a nucleic acid decomposition enzyme contained in the liquid by diluting the liquid containing the nucleic acid with the solution containing the ionic surfactant. In particular, in a case where the nucleic acid contained in the liquid is RNA, there is a concern that the RNA is decomposed by RNase A and the reverse transcription reaction is not performed. For this reason, it is preferable that the method of preparing a reagent include the dilution procedure S0 prior to the dissolution procedure S1. In addition, it is preferable that the method of preparing a reagent include the dilution procedure S0 even in a case where the nucleic acid contained in the liquid is a bacterial genome or the like. This is because it is possible to simply perform bacteriolysis in a heating procedure to be described.

It is possible to exhibit the above-described effect by adding the ionic surfactant such as SDS to the liquid containing the nucleic acid. On the other hand, there is a concern that the ionic surfactant in the nucleic acid amplification reaction inhibits the activity of the DNA polymerase and deteriorates the efficiency of the nucleic acid amplification reaction. Since the solid phase reagent according to an embodiment of the present technology contains the cyclodextrin, it is possible to include the ionic surfactant even in a case where the liquid contains the ionic surfactant. Accordingly, it is difficult to inhibit the nucleic acid amplification reaction even in a case where the liquid contains the ionic surfactant. In order to exhibit the effect of the inclusion of the ionic surfactant due to the cyclodextrin, for example, it is preferable that the concentration of the cyclodextrin be higher than or equal to 8 times the concentration of the sodium dodecyl sulfate (refer to Example 6).

In the method of preparing a sample according to an embodiment of the present technology, it is possible to use the ionic surfactant for reducing the decomposition of the nucleic acid or for extracting a genome from the cell such as the bacterial genom. In addition, it is possible to securely perform the nucleic acid amplification reaction using the cyclodextrin contained in the solid phase reagent even in a case of using the ionic surfactant. Accordingly, it is possible to simply and securely perform the nucleic acid amplification reaction using the method of preparing a sample according to an embodiment of the present technology.

### (3) Amplification Procedure

In amplification procedure S2, the nucleic acid contained in the liquid is amplified using the liquid in which the solid phase reagent is dissolved in the above-described dissolution procedure S1. In the amplification procedure S2, it is possible to appropriately select a method from existing nucleic acid amplification methods to perform the nucleic acid amplification reaction. Examples of the nucleic acid amplification method include polymerase chain reaction (PCR) that implements a temperature cycle. In addition, various isothermal amplification methods which are not accompanied with the temperature cycle may also be used. Examples of the isothermal amplification method include a Loop-mediated Isothermal Amplification (LAMP) method and a Transcription-Reverse transcription Concerted (TRC) method. In the nucleic acid amplification method according to an embodiment of the present technology, an isothermal amplification method that isothermally performs the amplification of the nucleic acid may be preferable, for example, the LAMP method is preferable as the isothermal amplification method.

### (4) Reverse Transcription Reaction Procedure

The method of preparing a sample may include a procedure of performing reverse transcription reaction using a ribonucleic acid as a template prior to the amplification procedure S2 in a case where the nucleic acid contained in the liquid is the ribonucleic acid (RNA). The reverse transcription reaction and the nucleic acid amplification reaction may be performed separately or may be continuously performed in a reaction site, for example, performing reverse transcription reaction-polymerase chain reaction (RT-PCR) or performing reverse transcription reaction-LAMP (RT-LAMP). In a case of continuously performing the two reactions, it is preferable that the solid phase reagent contain a reverse transcriptase in addition to the DNA polymerase.

### (5) Sonication Procedure

The method of preparing a sample according to an embodiment of the present technology may include a sonication procedure of the diluted solution of the liquid prior to the dissolution procedure S1. The sonication procedure is optional in the method of preparing a sample according to an embodiment of the present technology. However, for example, in a case where there is a nucleic acid as a template in a cell similarly to a case of the bacterial genome, it is possible to break the cell membrane by performing the sonication and to ease the release of the nucleic acid in the diluted solution. For this reason, the nucleic acid amplification reaction becomes more efficient as the nucleic acid can easily contact the primer or other components of the reagent compared to a case where the nucleic acid remains in the cell.

In the sonication procedure of the diluted solution, an existing ultrasonic generator can be used. For example, a contact-type ultrasonic generator such as a horn-type ultrasonic homogenizer can be used. In addition, a non-contact-type ultrasonic device which does not come in contact with a sample can also be used. The frequency of the ultrasonic wave can be appropriately selected based on the performance of the ultrasonic generator or the property of the liquid.

### (6) Heating Procedure

The method of preparing a sample according to an embodiment of the present technology may include a procedure of heating the diluted solution of the liquid prior to the dissolution procedure S1. The dissolution procedure is optional in the method of preparing a sample according to an embodiment of the present technology. However, for example, in a case where there is a nucleic acid as a template in a cell similarly to a case of the bacterial genome, as with the sonication, it is possible to perform bacteriolysis by heating the diluted solution. In addition, even if the nucleic acid as a template is from a virus, for example, in a case where the virus has an envelope, it is possible to separate a viral genome from the envelope and to diffuse the viral genome in the liquid using the heating procedure.

When performing the heating procedure, it is preferable that the viral genome be diluted in a liquid containing an ionic surfactant such as SDS in the dilution procedure S0 in order to more securely perform the dissolution of the cell membrane or the separation of the envelope as described above. The concentration of the SDS in the diluted solution in the heating procedure is preferably higher than or equal to 0.01% and less than 1%, and more preferably higher than or equal to 0.1% and less than 1% (refer to Example 5).

### 3. Microchip

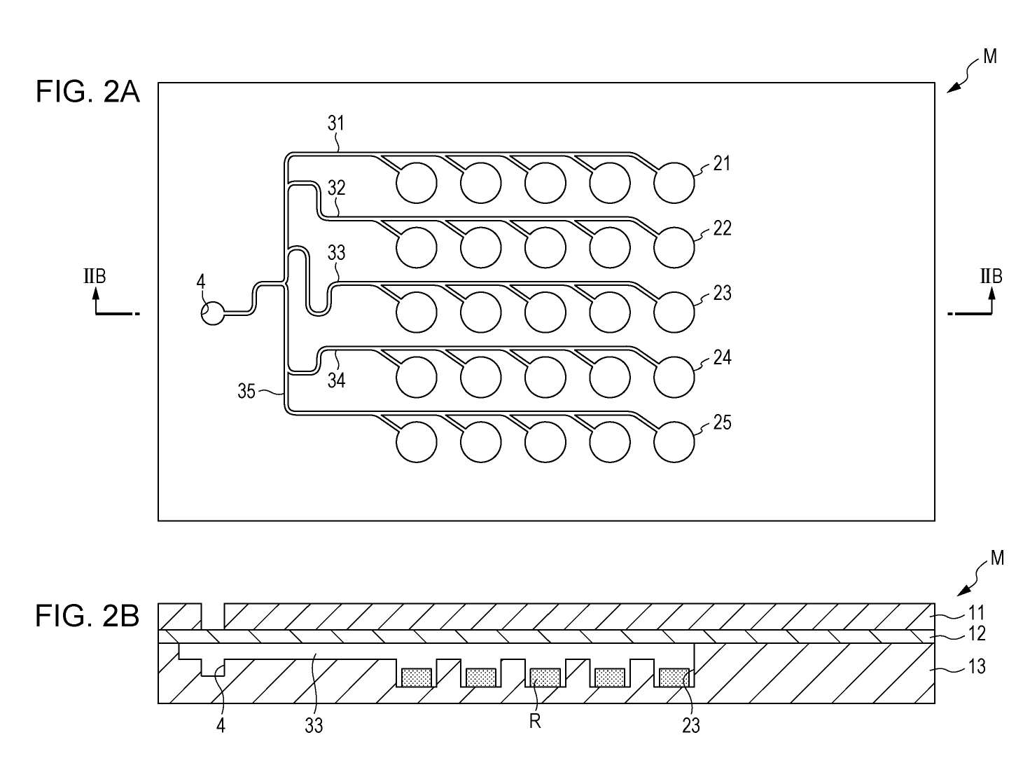

The solid phase reagent according to an embodiment of the present technology described above is suitable for nucleic acid amplification reaction using a microchip. FIGS. 2A and 2B illustrate a microchip according to a first embodiment of the present technology. FIG. 2A is a top view of a microchip M and FIG. 2B is a cross-sectional view taken along line IIB-IIB of an arrow of FIG. 2A. The microchip M is configured to have three substrate layers 11, 12 and 13 (refer to FIG. 2B). In addition, the microchip M is provided with wells 21 to 25 as reaction sites of a plurality of nucleic acid amplification reactions. In FIGS. 2A and 2B, five wells that communicate with a flow channel are allocated with an identical reference numeral.

The microchip M is provided with a reagent for solid phase nucleic acid amplification reaction (solid phase reagent) R at least containing DNA polymerase, cyclodextrin, and a binder. As shown in FIG. 2B, it is preferable that the solid phase reagent R be provided in a plurality of reaction sites (wells 23) installed in the microchip M. In addition, as shown in FIG. 2A, the reaction sites (wells 21 to 25) communicate with an entrance 4 through which a liquid enters the microchip M, through flow channels 31 to 35. The shape of the microchip M according to an embodiment of the present technology is not limited to the shape shown in FIGS. 2A and 2B, and the number of wells 21 to 25, or the like can be appropriately designed based on the purpose of utilization of the microchip.

The microchip M according to a first embodiment of the present technology is provided with a reagent for solid phase nucleic acid amplification reaction at least containing the DNA polymerase, cyclodextrin, and a binder in the inside thereof. For this reason, it is possible to prepare the sample for nucleic acid amplification reaction in the microchip by the entry of the liquid containing the nucleic acid from the outside.

In addition, since the solid phase reagent R contains the cyclodextrin, it is possible to suppress the deterioration of the activity of the DNA polymerase contained in the solid phase reagent R and to securely perform the nucleic acid amplification reaction in the microchip M.

Furthermore, since the solid phase reagent R contains the binder, the shape of the reagent is stabilized. For this reason, even in a case where the solid phase reagent R is provided in a plurality of reaction sites (wells 21 to 25), the shape of the solid phase reagent R is uniform. Therefore, it is possible to suppress the variation regarding the dissolution of the solid phase reagent R caused by the liquid entered the microchip M. In addition, it is possible to align the timing of the start of the nucleic acid amplification reaction by providing the solid phase reagent R in the reaction sites of the nucleic acid amplification reaction compared to a case where a sample for nucleic acid amplification reaction is prepared and subsequently enters the reaction sites. Accordingly, it is possible to accurately improve the nucleic acid amplification reaction performed using the microchip M.

Embodiments of the present technology can be configured as the following.

(1) A method of preparing a sample for nucleic acid amplification reaction, including: a procedure of dissolving a solid phase reagent at least containing DNA polymerase, cyclodextrin, and a binder, in a liquid containing a nucleic acid.

(2) The method of preparing a sample for nucleic acid amplification reaction according to above-described (1), further including: a procedure of diluting the liquid with a solution containing an ionic surfactant prior to the procedure of the dissolving of the solid phase reagent.

(3) The method of preparing a sample for nucleic acid amplification reaction according to above-described (2), in which the ionic surfactant is an anionic surfactant.

(4) The method of preparing a sample for nucleic acid amplification reaction according to above-described (3), in which the anionic surfactant is sodium dodecyl sulfate.

(5) The method of preparing a sample for nucleic acid amplification reaction according to above-described (4), in which the concentration of the cyclodextrin is higher than or equal to 8 times the concentration of the sodium dodecyl sulfate.

(6) The method of preparing a sample for nucleic acid amplification reaction according to above-described (2) to (5), further including: a procedure of performing sonication of the diluted solution of the liquid prior to the procedure of the dissolving of the solid phase reagent.

(7) The method of preparing a sample for nucleic acid amplification reaction according to above-described (2) to (5), further including: a procedure of heating the diluted solution of the liquid prior to the procedure of the dissolving of the solid phase reagent.

(8) A nucleic acid amplification method including: a procedure of dissolving a solid phase reagent at least containing DNA polymerase, cyclodextrin, and a binder, in a liquid containing a nucleic acid; and a procedure of amplifying the nucleic acid.

(9) The nucleic acid amplification method according to above-described (8), in which amplification of the nucleic acid is isothermally performed.

(10) The nucleic acid amplification method according to above-described (8) or (9), in which the nucleic acid is the ribonucleic acid and the nucleic acid amplification method further includes a procedure of performing reverse transcription reaction using a ribonucleic acid as a template prior to the procedure of the amplifying of the nucleic acid.

(11) A reagent for solid phase nucleic acid amplification reaction including: at least a DNA polymerase, cyclodextrin, and a binder.

(12) The reagent for solid phase nucleic acid amplification reaction according to above-described (11), in which the cyclodextrin includes a hydroxypropyl group.

(13) The reagent for solid phase nucleic acid amplification reaction according to above-described (11) or (12), in which the reagent for solid phase nucleic acid amplification reaction is mixed in a liquid containing a template nucleic acid chain and an ionic surfactant.

(14) The reagent for solid phase nucleic acid amplification reaction according to above-described (12) and (13), in which the concentration of the cyclodextrin is higher than or equal to 8 times the concentration of the ionic surfactant.

(15) The reagent for solid phase nucleic acid amplification reaction according to above-described (11) to (14), further including ribonuclease H.

(16) A microchip including a reagent for solid phase nucleic acid amplification reaction at least containing DNA polymerase, cyclodextrin, and a binder.

(17) The microchip according to above-described (16), in which the reagent for solid phase nucleic acid amplification reaction is provided in each of a plurality of reaction sites of nucleic acid amplification reaction installed in the microchip, and the reaction sites communicate with an entrance through which a liquid enters the microchip, through flow channels.

### EXAMPLE

### Example 1

### 1. Verification of Maintenance of Activity of Sample for Nucleic Acid Amplification Reaction Due to Cyclodextrin

It was verified whether the activity of a sample for nucleic acid amplification reaction is maintained by adding cyclodextrin to the sample for nucleic acid amplification reaction containing DNA polymerase.

### Material and Method

The composition of a solid phase reagent used in Example 1 is shown in Table 1. As the cyclodextrin, hydroxyprophyl-β-cyclodextrin (HPβCD) was used in Example 1. As the binder, a binder solution in which any one or more of sucrose, dextran, polyethylene glycol (PEG), trehalose, collagen peptides, gelatin, BSA, FICOLL, and polyvinyl pyrrolidone is/are dissolved was used. Bst DNA polymerase Lg Frag (NEW ENGLAND BIOLABS) was used as the DNA polymerase. A reagent solution containing the HPβCD, the binder solution, and the DNA polymerase was mixed so as to become a predetermined concentration shown in Table 1, the mixture was dispensed to containers and was freeze-dried to obtain solid phase reagents of Test Examples 1 to 6. In addition, as Comparative Example 1, a reagent solution containing neither HPβCD nor binder was also prepared and freeze-dried. Furthermore, as Comparative Example 2, a reagent solution that does not contain a binder and was mixed with HPβCD, and the mixture was freeze-dried.

TABLE 1

| | Comparative | | | | | | | |
|---|---|---|---|---|---|---|---|---|
| | Example | | Test Example | | | | | |
| | 1 | 2 | 1 | 2 | 3 | 4 | 5 | 6 |
| HPβCD concentration | 0.0 | 3.6 | 3.0 | 3.0 | 3.6 | 3.6 | 3.6 | 3.6 |
| (w/v %) | | | | | | | | |
| Binder concentration | 0.0 | 0.0 | 5.7 | 5.7 | 4.2 | 4.8 | 4.8 | 5.6 |
| (w/v %) | | | | | | | | |
| Time until the reaction | No reaction | +17 | +10 | +36 | +23 | +60 | +3 | +11 |
| starts (%) | | | | | | | | |
| Solid Phase State | X | X | ○ | ○ | ○ | ○ | ○ | Δ |

Above-described Comparative Examples 1 and 2 and Test Examples 1 to 6 were dissolved in a liquid containing a template nucleic acid to perform nucleic acid amplification reaction using a LAMP method. An amplification nucleic acid chain was detected using a quenching probe that specifically hybridizes with the amplification nucleic acid chain is performed. The end of the quenching probe is bonded with a fluorescent material. In the quenching probe, the bonded fluorescent material emits if the quenching probe is not hybridized with the nucleic acid chain but the fluorescent material is quenched if the quenching probe is hybridized with the nucleic acid. It is possible to detect the amplification of the nucleic acid by measuring the fluorescent variation.

### Result

The result of Example 1 is shown in Table 1. In Table 1, the time until the reaction starts indicates the time until the nucleic acid amplification reaction starts, correspond to a Tt value (minute), and is based on an inflexion point of a quenching signal of the fluorescent material. Regarding the time which is necessary until the time where it is determined that the nucleic acid amplification reaction was started using the LAMP method, the increase with respect to the time (Tt value) as a reference when the nucleic acid amplification reaction was performed using a reagent that constitutes an identical composition and that is not freeze-dried, is indicated with a ratio (%). In addition, the solid phase state indicates a state of a reagent after the freeze-drying. “X” indicates that the reagent did not become the solid phase state, “Δ” indicates that the reagent became the solid phase state, and “◯” indicates that the solid phase state was maintained over a long period.

As shown in Table 1, amplification of the nucleic acid could not be confirmed in Comparative Example 1 containing no HPβCD. On the other hand, amplification of the nucleic acid was performed in Comparative Example 2 and Test Examples 1 to 6 that contain HPβCD, even though it takes longer time until the start of the nucleic acid amplification reaction compared to a case where the reagent is not freeze-dried. From the result, it was confirmed that the activity of the DNA polymerase was maintained even with the freeze-dried reagent when the HPβCD was added.

In addition, as shown in Table 1, it was difficult to make the reagent be a solid phase even if the reagent is freeze-dried in Comparative Example 2 containing no binder. On the other hand, it was confirmed that the reagent became a solid phase in Test Examples 1 to 6 containing a binder. From the above results, it was confirmed that the cyclodextrin and the binder are necessary to maintain the activity of DNA polymerase contained in the reagent and to maintain the state of the reagent to be a solid phase.

### Example 2

### 2. Verification of Effect of Cyclodextrin on Nucleic Acid Amplification Reaction Solution to which SDS is Added

In Example 2, it was verified as to whether the influence on the nucleic acid amplification reaction of the SDS is deteriorated by adding the cyclodextrin.

### Material and Method

The composition of a reagent used in Example 2 is shown in Table 2. As the cyclodextrin, hydroxyprophyl-β-cyclodextrin (HPβCD) was used in Example 2. RT-LAMP reaction reagents to which the SDS and the cyclodextrin are added with a predetermined concentration were set as Examples 1 to 3. In addition, only an RT-LAMP reaction reagent was prepared as Comparative Example 1. Furthermore, an LAMP reaction reagent containing SDS with a concentration of 0.4% was set as Comparative Example 2. The above-described Comparative Examples 1 and 2 and Test Examples 1 to 3 are respectively mixed with template nucleic acids to perform the nucleic acid amplification reaction using the LAMP method. An amplification nucleic acid chain was detected using the quenching probe similarly to Example 1.

### Result

The result of Example 2 is shown in Table 2. In Table 2, the time until the reaction starts is as described in Example 1. As shown in Table 2, it was confirmed that the nucleic acid amplification reaction is inhibited by adding the SDS to the nucleic acid amplification reaction solution (Comparative Example 2). In addition, it was confirmed that the nucleic acid amplification reaction is inhibited even if the HPβCD is added with a concentration of 5 times the SDS (Test Example 1). On the other hand, the amplification of the nucleic acid was confirmed in Test Example 2 containing the HPβCD with a concentration of 10 times the SDS and in Test Example 3 containing the HPβCD with a concentration of 15 times the SDS. From the above results, it was confirmed that, even in presence of SDS, the nucleic acid amplification reaction is not inhibited by adding the cyclodextrin to the nucleic acid amplification reaction solution with a concentration of about 10 times the SDS, for example.

TABLE 2

| | Compar- | Compar- | | | |
|---|---|---|---|---|---|
| | ative | ative | Test | Test | Test |
| | Example 1 | Example 2 | Example 1 | Example 2 | Example 3 |
| SDS | 0.0 | 0.4 | 0.4 | 0.4 | 0.4 |
| concen- | | | | | |
| tration | | | | | |
| (w/v %) | | | | | |
| HPβCD | 0.0 | 0.0 | 2.0 | 4.0 | 6.0 |
| concen- | | | | | |
| tration | | | | | |
| (w/v %) | | | | | |
| Reaction | 12.7 | No | No | 11.7 | 11.8 |
| time | | reaction | reaction | | |
| (minute) | | | | | |

### Example 3

### 3. Verification of Suppression of Activity of RNase A Due to SDS

It was verified as to whether the activity of RNase A contained in the solution is suppressed by adding the SDS to the solution.

### Material and Method

RNase Alert QC test Kit (Ambion) was used for measuring the activity of the RNase A. The RNase A was added to the solution containing RNaseAlert substrate with a final concentration of 0.003 U/mL. The SDS was added to the RNase A solution with a final concentration of 0.1% or 1.0% each to be set as Test Example 1 and Test Example 2. In addition, a solution containing neither RNase A nor SDS was set as Comparative Example 1 and a solution that contains the RNase A but does not contain the SDS was set as Comparative Example 2. Test Examples 1 and 2 and Comparative Examples 1 and 2 were kept warm for 60 minutes at a temperature of 37° C. A fluorescent material (FAM) is bonded with a quencher in the RNaseAlert substrate. The fluorescent material emits when the fluorescent material is separated from the quencher using the RNase A. Excitation light and luminescence of the fluorescence was measured to be 490 nm and 520 nm respectively, using Chromo4 (Bio-rad).

### Result

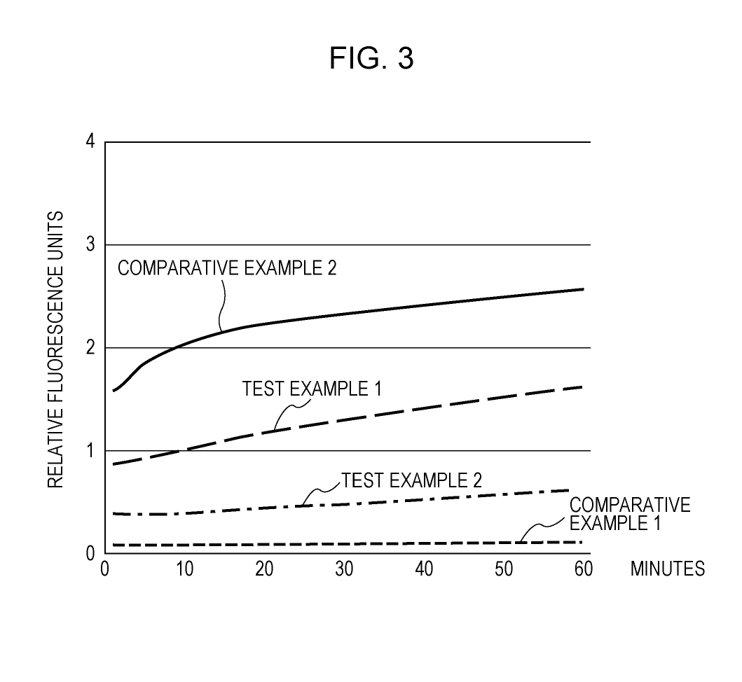

The result of Example 3 is shown in FIG. 3. The vertical axis of FIG. 3 indicates the fluorescence intensity (relative fluorescence units) and the horizontal axis indicates the time. As shown in FIG. 3, the increase of the fluorescence intensity was confirmed in Comparative Example 2 to which the RNase A is added but the SDS is not added, that is, the activity of the RNase A is not inhibited. On the other hand, in Comparative Example 1 to which neither RNase A nor SDS is added, increase of the fluorescence intensity was not confirmed, that is, there is no activity of the RNase A.

In addition, in Test Example 1 and Test Example 2 to which the RNase A and the SDS were added, the activity of the RNase A was suppressed compared to Comparative Example 2. Furthermore, there was a tendency that the activity of the RNase A was suppressed in Test Example 2 having a higher concentration of the SDS. From the results, it was confirmed that the activity of the RNase A can be suppressed by the SDS.

### Example 4

### 4. Verification of Effect of Suppressing Activity of RNase A Contained in Organism-Derived Sample Due to SDS

In Example 4, it was verified as to whether the SDS is effective for suppressing the activity of the RNase A contained in an organism-derived sample.

### Material and Method

In Example 4, bovine plasma was used as an organism derived sample. The bovine plasma was used after being diluted 10 times or 20 times in advance. Similarly to Example 3, RNase Alert QC test Kit (Ambion) was used for measuring the activity of the RNase A. RNaseAlert substrate and SDS were added to the 10 times diluted bovine plasma such that final concentrations of the SDS become 0.05%, 0.1%, 0.2%, 0.3%, 0.4%, and 0.5% respectively to be set as Test Examples 1 to 6. In addition, RNaseAlert substrate and SDS were added to the 20 times diluted bovine plasma such that final concentrations of the SDS become 0.05%, 0.1%, 0.2%, 0.3%, 0.4%, and 0.5% respectively to be set as Test Examples 7 to 12. In addition, 10 times diluted bovine plasma to which SDS is not added was set as Comparative Example 1 and 20 times diluted bovine plasma to which SDS is not added was set as Comparative Example 2. Test Examples 1 to 12 and Comparative Examples 1 and 2 were kept warm for 60 minutes at a temperature of 37° C. and the activity of the RNase A was measured during the time period using the same method as in Example 3.

### Result

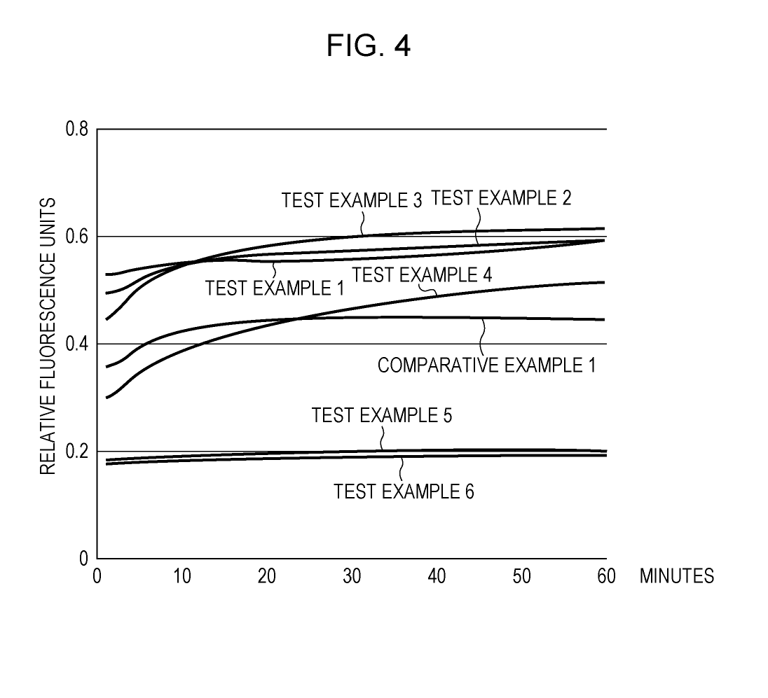

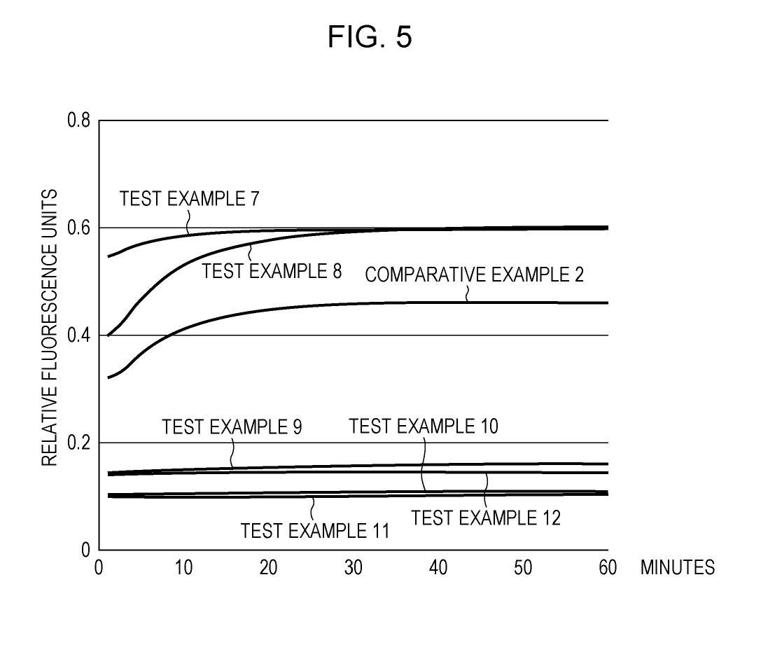

The result of Example 4 is shown in FIGS. 4 and 5. The vertical axes of FIGS. 4 and 5 indicate the fluorescence intensity (relative fluorescence units) and the horizontal axes indicate the time. FIG. 4 shows the result of Comparative Example 1 and Test Examples 1 to 6 and FIG. 5 shows the result of Comparative Example 2 and Test Examples 7 to 12.

As shown in Test Examples 5 and 6 of FIG. 4, in a case where the bovine plasma was diluted 10 times, the activity of the RNase A was suppressed with the SDS concentration higher than or equal to 0.4%. On the other hand, as shown in Test Examples 9 to 12 of FIG. 5, in a case where the bovine plasma was diluted 20 times, the activity of the RNase A was suppressed with the SDS concentration higher than or equal to 0.2%. From the above-described results, it was confirmed that it is possible to suppress the activity of the RNase A containing the organism-derived sample by adding the SDS. In addition, the dilution ratio and the SDS concentration of the organism-derived sample necessary for suppressing the activity of the RNase A can be represented by the following formula.

[SDS concentration %]≧0.02×[Dilution rate of organism sample]+0.6

### Example 5

### 5. Review of SDS Concentration Necessary for Extracting Nucleic Acid from Bacteria

In Example 5, the concentration of SDS necessary for extracting a nucleic acid from a fungus body was reviewed for a nucleic acid amplification reaction.

### Material and Method

In Example 5, Bifidobacterium bifidum from which it is comparatively difficult to extract the nucleic acid was used as bacteria. The Bifidobacterium bifidum (NBRC number: 100015) was obtained from Biological Resource Center (NBRC) of National In statute of Technology and Evaluation (NITE). In addition, the nucleic acid amplification reaction of a genome of Bifidobacterium bifidum was performed using the LAMP method and an LAMP reaction reagent (Eiken Chemical Co., Ltd, Loopamp® DNA Amplification reagent Kit) was used as a sample for the nucleic acid amplification reaction. In addition, five types of primers shown in Table 3 were used.

TABLE 3

| | | Sequence |
|---|---|---|
| Primer or probe | Base sequence | number |
| Primer F3 | TGCTCCGGAA TAGCTCCTG | 1 |
| Primer B3 | TGCCTCCCGT AGGAGTCT | 2 |
| Primer FIP | CCAACAAGCT GATAGGACGC | 3 |
| | GACGCATGTG ATTGTGGGAA | |
| | AG | |
| Primer BIP | GAGGTAACGG CTCACCAAGG | 4 |
| | CGCCGTATCT CAGTCCCAAT | |
| | G | |
| Primer LF | CCATCCCACG CCGATAG | 5 |
| Quenching probe | CCGGCCTGAG AGGGCGACC | 6 |

A quenching probe shown in Table 3 was used for detecting an amplification nucleic acid chain. In Example 5, excitation light and luminescence of light derived from a fluorescent material FAM bonded with the quenching probe was measured to be 490 nm and 520 nm respectively, using Chromo4 (Bio-rad).

A cell suspension in which the Bifidobacterium bifidum is prepared to be 100 copies/ml. In Example 5, as Test Examples 3 to 5, SDS was added to the cell suspension at the time of heat treatment such that concentrations of SDS become 0.01%, 0.1%, and 1%, respectively (refer to Table 4). In addition, as Test Examples 1 and 2 and Comparative Example 1, cell suspensions to which SDS was not added was prepared (refer to Table 4). The heat treatment was performed for 3 minutes at a temperature of 90° C. for Test Examples 1 and 3 to 5 before starting the nucleic acid amplification reaction. In addition, sonication was performed for Test Example 2 before starting the nucleic acid amplification reaction. After the heating process or the sonication, a LAMP reaction reagent and a primer and quenching probe of Table 3 were added to Test Examples 1 to 5. Furthermore, hydroxyprophyl-β-cyclodextrin (Tokyo Chemical Industry Co., Ltd.) was added to Test Examples 1 to 5 such that a final concentration of the hydroxyprophyl-β-cyclodextrin becomes 5% (w/v). A sample for nucleic acid amplification reaction, a primer, a quenching probe, and HPβCD were added to Comparative Example 1 as well. The nucleic acid amplification reaction was performed for 60 minutes at a temperature of 63° C.

TABLE 4

| | Comparative | Test | Test | Test | Test | Test |
|---|---|---|---|---|---|---|
| | Example 1 | Example 1 | Example 2 | Example 3 | Example 4 | Example 5 |
| SDS concentration | 0.00 | 0.00 | 0.00 | 0.01 | 0.10 | 1.00 |
| (w/v %) | | | | | | |
| Heat treatment | None | Yes | None | Yes | Yes | Yes |
| Sonication | None | None | Yes | None | None | None |

### Result

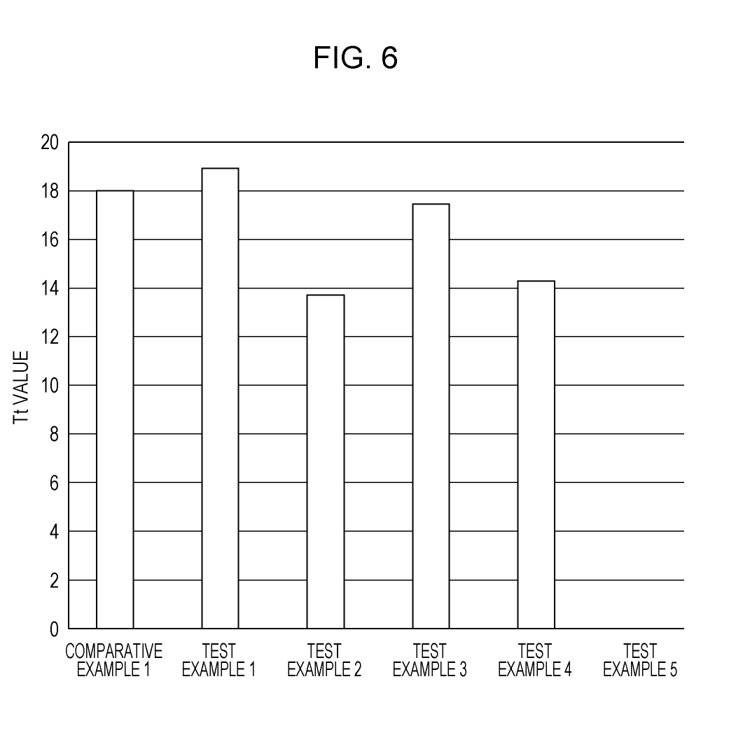

The result of Example 5 is shown in FIG. 6. FIG. 6 shows Tt values (minute) of Test Examples 1 to 5 and Comparative Example 1. The Tt value of Comparative Example 1 was 18.0. In addition, the Tt value of Test Example 2 to which sonication was performed was 13.7. This shows that the Tt value of Test Example 2 is lower than that of Comparative Example 1 and there are many genomes of Bifidobacterium bifidum as template nucleic acids in the nucleic acid amplification reaction solution. That is, it shows that the genomes are extracted from a fungus body.

On the contrary, the Tt values of Test Examples 1, 3, and 4 to which heat treatment was performed were 18.9, 17.5, and 14.2 respectively. In addition, there was no amplification of the nucleic acid in Test Example 5. It was confirmed that Test Example 4 in which the SDS concentration was 0.1% is low compared to that of Comparative Example 1 and the SDS concentration of Test Example 4 was almost the same as that of Test Example 2. In addition, it is considered that an inhibition of the nucleic acid amplification reaction occurs due to SDS in Test Example 5. From the above-described results, it can be seen that it is preferable to set the SDS concentration to be higher than or equal to 0.1% and less than 1.0% at the time of heat treatment under the condition where nucleic acid is extracted from a fungus body.

### Example 6

### 6. Review of Cyclodextrin Concentration Necessary for Suppressing Inhibition of Reaction due to SDS in Nucleic Acid Amplification Reaction

In Example 6, the concentration of cyclodextrin that can suppress the inhibition of the nucleic acid amplification reaction due to SDS was reviewed.

### Material and Method

In Example 6, nucleic acid amplification reaction was performed using the LAMP method using a genome of Bifidobacterium bifidum as a template nucleic acid. In addition, a sample for nucleic acid amplification reaction, a primer, and a quenching probe used for the nucleic acid amplification reaction were the same as those in Example 5. HPβCD was prepared such that a final concentration of HPβCD in a nucleic acid amplification reaction solution becomes 2.5%. Furthermore, SDS was added thereto such that the SDS concentrations become 0%, 0.05%, 0.1%, 0.2%, 0.3%, 0.4%, 0.5%, and 1.0% to be set as Test Examples 1 to 8 respectively. In addition, HPβCD was prepared such that a final concentration of HPβCD in a nucleic acid amplification reaction solution becomes 5.0%. Furthermore, SDS was added thereto such that the SDS concentrations become 0%, 0.05%, 0.1%, 0.2%, 0.3%, 0.4%, 0.5%, and 1.0% to be set as Test Examples 9 to 16 respectively. In addition, nucleic acid amplification reaction solutions prepared such that the HPβCD concentration becomes 0% and the SDS concentrations become 0%, 0.01%, and 0.1% to be set as Comparative Examples 1 to 3.

### Result

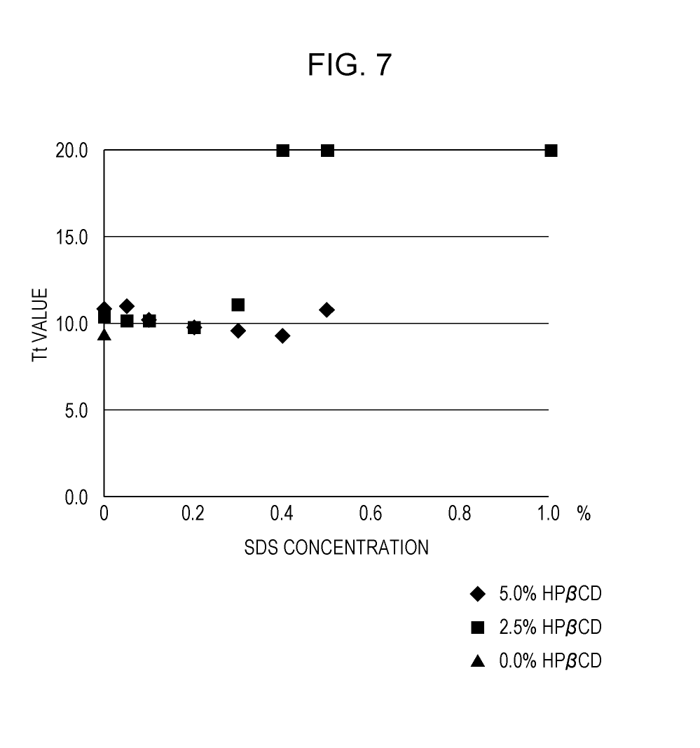

The result of Example 6 is shown in FIG. 7. When the HPβCD concentration is 2.5% at the time of the nucleic acid amplification reaction, it was possible to suppress the effect of inhibiting the nucleic acid amplification reaction due to the SDS with respect to the SDS concentrations between 0.0% and 0.3% (Test Examples 1 to 5). In addition, when the HPβCD concentration is 5.0% at the time of the nucleic acid amplification reaction, it was possible to suppress the effect of inhibiting the nucleic acid amplification reaction due to the SDS with respect to the SDS concentrations between 0.0% and 0.5% (Test Examples 9 to 15).

From the results of Example 6, it can be seen that the HPβCD concentration is preferably higher than or equal to 8 times the SDS concentration and is more preferably higher than or equal to 10 times the SDS concentration, in the nucleic acid amplification reaction solution in order to suppress the effect of inhibiting the LAMP reaction due to the SDS. In addition, even if the HPβCD is not added thereto, amplification of the nucleic acid was confirmed when the SDS concentration is lower than or equal to 0.01%. No amplification of the nucleic acid was confirmed when the HPβCD is not added and the SDS concentration is 0.1% (Comparative Examples 2 and 3 are not shown in the drawings).

### Example 7

### 7. Verification of Nucleic Acid Amplification Reaction Using Virus-derived Nucleic Acid as Template Nucleic Acid

In Example 7, amplification of a nucleic acid was attempted by performing nucleic acid amplification reaction using a nasal cavity swab derived from a patient infected with an influenza virus.

### Material and Method

A nasal cavity swab samples obtained from six patients infected with an influenza virus were respectively dissolved in sample dilution solutions of 4 ml (20 mMTris-HCl, 0.2% SDS). Each of the sample dilution solutions of 10 μL after the dissolution of the nasal cavity swab samples was mixed with an RT-LAMP nucleic acid amplification reagent (Loopamp® RNA Amplification reagent Kit (Eiken Chemical Co., Ltd)) to prepare a nucleic acid amplification reaction solution (25 μL) and each prepared nucleic acid amplification reaction solution was set as Test Examples 1 to 6. In addition, an existing primer was used as a primer for the LAMP reaction with respect to influenza virus type A in Example 7 (refer to J Med Virol, January 2011, 83(1):10-15). The reaction condition of the nucleic acid amplification reaction and the method of detecting the amplification nucleic acid chain are the same as that of Example 5.

### Result

The result of Example 5 is shown in Table 5. Table 5 shows Tt values (minute) of Test Examples 1 to 6. As shown in Table 5, amplification of the nucleic acid was confirmed in all of Test Examples 1 to 6. From the above-described result, it was confirmed that it is possible to perform the nucleic acid amplification reaction using the influenza virus as a template which has enveloped by diluting the liquid containing a nucleic acid with a saluting containing SDS as an ionic surfactant.

TABLE 5

| Test | Tt value |
|---|---|
| Example | (minute) |
| 1 | 7.5 |
| 2 | 8.9 |
| 3 | 11.4 |
| 4 | 11.1 |
| 5 | 17.2 |
| 6 | 14.9 |

### Example 8

### 8. Verification of Amplification of Virus-Derived Nucleic Acid According to Nucleic Acid Amplification Reaction Using Solid Phase Reagent

In Example 8, it was verified as to whether it is possible to amplify a virus-derived nucleic acid using a solid phase reagent similarly to the liquid phase reagent.

### Material and Method

In Example 8, nucleic acid amplification reaction was performed using the LAMP method by replacing the liquid phase sample for nucleic acid amplification reaction in Example 7 with a solid phase sample for nucleic acid amplification reaction. The solid phase sample for nucleic acid amplification reaction used in Example 8 contains Bst DNA polymerase Lg Frag (NEW ENGLAND BIOLABS) as DNA polymerase. In addition, the solid phase reagent contains ThermoScript (Life technologies) as reverse transcriptase in which the RNase H activity is suppressed. Hybridase Thermostable RNase H (EPICENTRE) was used as RNase H. Furthermore, the solid phase reagent contains the HPβCD, and the binder described in Example 1. Nasal cavity swab samples derived from the 6 patients infected with the influenza virus were used similarly to Example 7. Respective nasal cavity swabs are dissolved in sample dilution solutions of 10 ml (20 mMTris HCl, 0.2% SDS) to be set as Test Examples 1 to 6. The sample dissolution liquid, the above-described solid phase reagent, a primer, and a quenching probe were mixed to perform nucleic acid amplification reaction using the RT-LAMP method. The RT-LAMP reaction was performed without a process of diluting the sample dissolution liquid with a reagent solution because the solid phase reagent was used in Example 8. The reaction condition of the nucleic acid amplification reaction and the method of detecting the amplification nucleic acid chain are the same as that of Example 5.

In Example 8, amplification of the nucleic acid was confirmed in all of Test Examples 1 to 6. That is, it was confirmed that it is possible to perform amplification of the nucleic acid and to detect a virus-derived genome contained in the sample using the solid phase reagent.

### Example 9

### 9. Verification of Effect of RNase H in Nucleic Acid Amplification Reaction Including Reverse Transcription Reaction

In Example 9, the effect of RNase H was verified in nucleic acid amplification reaction including reverse transcription reaction using RNA as a template.

### Material and Method

In Example 9, ThermoScript (Life technologies) was used as a reverse transcriptase in which an RNase H activity is suppressed. In addition, Bst DNA polymerase LG Frag (NEW ENGLAND BIOLABS) was used as the DNA polymerase. Furthermore, Hybridase Thermostable RNase H (EPICENTRE) was used as RNase H. The reverse transcriptase (3.75 U/25 μL), DNA polymerase (16 U/25 μL), a primer, a quenching probe, and a template nucleic acid (RNA) were mixed to each other. Samples 4 to 6 were prepared such that the RNase H is added to the mixed solution so as to be 0.63 U/25 μL of a concentration of the RNase H, and were set as Test Group 1. In addition, mixed solutions to which RNase H was not added (Samples 1 to 3) were also prepared and were set as Comparative Group 1. The nucleic acid amplification reaction in Test Group 1 and Comparative Group 1 was performed using the LAMP method. The reaction condition of the nucleic acid amplification reaction and the method of detecting the amplification nucleic acid chain are the same as that of Example 5.

### Result

The result of Example 9 is shown in Table 6. As shown in Table 6, there was amplification of the nucleic acid in Test Group 1 whereas there was no amplification of the nucleic acid in Comparative Group 1. From the result of Example 9, it can be seen that it is preferable to add RNase H in the nucleic acid amplification reaction accompanied with reverse transcription reaction.

TABLE 6

| | Comparative Group 1 | | | Test Group 1 | | |
|---|---|---|---|---|---|---|
| | Sample 1 | Sample 2 | Sample 3 | Sample 4 | Sample 5 | Sample 6 |
| RNase H | 0 | 0 | 0 | 0.63 | 0.63 | 0.63 |
| concentration | | | | | | |
| (U/25 μL) | | | | | | |
| Tt value | No reaction | No reaction | No reaction | 11.9 | 13.1 | 15.5 |
| Tt value | No reaction | | | 13.5 | | |
| (average) | | | | | | |

### Example 10

### 10. Review of RNase H Concentration in Nucleic Acid Amplification Reaction Accompanied with Reverse Transcription Reaction

In Example 10, the concentration of RNase H in nucleic acid amplification reaction accompanied with reverse transcription reaction was reviewed.

### Material and Method

In Example 10, Cloned AMV Reverse Transcriptase (Life technologies) was used as a reverse transcriptase having an RNase H activity. The same DNA polymerase and RNase H as those in Example 9 were used. RNase H was added to mixed solutions such that the concentrations of RNase H become 0.16 U/25 μL, 0.31 U/25 μL, and 0.63 U/25 μL to be set as Test Groups 1 to 3. In addition, mixed solutions to which RNase H was not added were also prepared and were set as Comparative Group 1. The nucleic acid amplification reaction in Test Groups 1 to 3 and Comparative Group 1 was performed using the LAMP method. The reaction condition of the nucleic acid amplification reaction and the method of detecting the amplification nucleic acid chain are the same as that of Example 5.

### Result

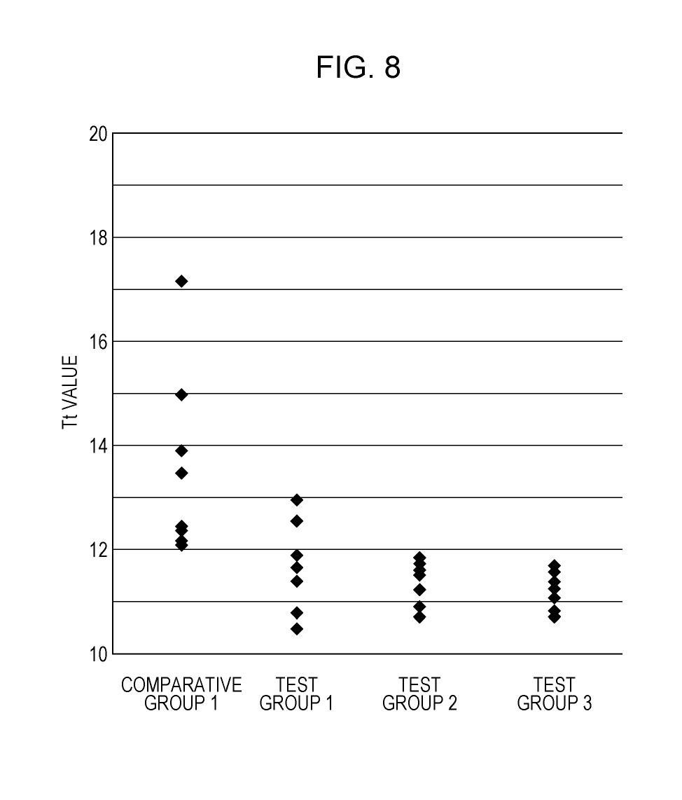

The result of Example 10 is shown in FIG. 8. FIG. 8 shows respective Tt values of Test Groups 1 to 3 and Comparative Group 1. As shown in FIG. 8, while there was variation of the Tt values in Comparative Group 1, the variation was suppressed in Test Group 1. In addition, the variation was further suppressed by increasing the amount of RNase H added to Test Groups 2 and 3. From the result of the Example 10, it can be seen that it is preferable that the concentration of the RNase H be higher than or equal to 0.16 U/25 μL in a case of using reverse transcriptase having an RNase H activity. In addition, it was confirmed that, even in a case of using the reverse transcriptase having the RNase H activity, the variation of the Tt values was suppressed and the Tt value became smaller in a case of Test Groups 1 to 3 to which an RNase H enzyme was added compared to a case of Comparative Group 1 to which RNase H was not added, thereby effectively performing the nucleic acid amplification reaction.

### Example 11

### 11. Effect on Nucleic Acid Amplification Reaction Due to RNase H Under Presence of RNase A Inhibitor

In Example 11, it was verified as to whether the effect on the nucleic acid amplification reaction confirmed in Example 9 is also exhibited in a sample to which an RNase A inhibitor was added.

### Material and Method

In Example 11, Ribonuclease Inhibitor (TAKARA BIO INC.) was used as an RNase A inhibitor. The same reagents and template nucleic acid chain other than the Ribonuclease Inhibitor are used as in Example 9. RNase H having a concentration of 0.63 U/25 μL was used. In addition, Samples 4 to 6 to which the RNase A inhibitor was not added were set as Test Group 1 and Samples 7 to 9 to which the RNase A inhibitor was added at a concentration of 25 U/25 μL were set as Test Group 2. Furthermore, Samples 1 to 3 to which neither RNase H nor RNase A inhibitor was added were set as Comparative Group 1. The nucleic acid amplification reaction in Test Group 1 and 2 and Comparative Group 1 was performed using the LAMP method. The reaction condition of the nucleic acid amplification reaction and the method of detecting the amplification nucleic acid chain are the same as that of Example 5.

### Result

The result of Example 11 is shown in Table 7. As shown in Table 7, it was confirmed that there is amplification of the nucleic acid in Test Groups 1 and 2 to which the RNase H was added. On the other hand, it was not confirmed that there is amplification of the nucleic acid in Comparative Group 1 to which the RNase H was not added. From the above, it was confirmed that the RNase H activity is not inhibited even by the addition of the RNase A inhibitor and it is possible to more effectively perform the nucleic acid amplification reaction using RNA as a template by the operation of the RNase H.

TABLE 7

| | Comparative Group 1 | | | Test Group 1 | | | Test Group 2 | | |
|---|---|---|---|---|---|---|---|---|---|
| | Sample 1 | Sample 2 | Sample 3 | Sample 4 | Sample 5 | Sample 6 | Sample 7 | Sample 8 | Sample 9 |
| RNase H concentration (U/25 μL) | 0 | 0 | 0 | 0.63 | 0.63 | 0.63 | 0.63 | 0.63 | 0.63 |
| RNase A inhibitor (U/25 μL) | 0 | 0 | 0 | 0 | 0 | 0 | 25 | 25 | 25 |
| Tt value | No | No | No | 11.9 | 13.1 | 15.5 | 14.1 | 11.2 | 15.0 |
| | reaction | reaction | reaction | | | | | | |

| | | | |
|---|---|---|---|
| Tt value (average) | No reaction | 13.5 | 13.4 |

It should be understood by those skilled in the art that various modifications, combinations, sub-combinations and alterations may occur depending on design requirements and other factors insofar as they are within the scope of the appended claims or the equivalents thereof.

What is claimed is:

1. A method of preparing a sample for nucleic acid amplification reaction, comprising:
a procedure of dissolving a solid phase reagent at least containing DNA polymerase, cyclodextrin, and a binder, in a liquid containing a nucleic acid.

2. The method of preparing a sample for nucleic acid amplification reaction according to claim 1, further comprising:
a procedure of diluting the liquid with a solution containing an ionic surfactant prior to the procedure of the dissolving of the solid phase reagent.

3. The method of preparing a sample for nucleic acid amplification reaction according to claim 2,
wherein the ionic surfactant is an anionic surfactant.

4. The method of preparing a sample for nucleic acid amplification reaction according to claim 3,
wherein the anionic surfactant is sodium dodecyl sulfate.

5. The method of preparing a sample for nucleic acid amplification reaction according to claim 4,
wherein the concentration of the cyclodextrin is higher than or equal to 8 times the concentration of the sodium dodecyl sulfate.

6. The method of preparing a sample for nucleic acid amplification reaction according to claim 2, further comprising:
a procedure of performing sonication of the diluted solution of the liquid prior to the procedure of the dissolving of the solid phase reagent.

7. The method of preparing a sample for nucleic acid amplification reaction according to claim 2, further comprising:
a procedure of heating the diluted solution of the liquid prior to the procedure of the dissolving of the solid phase reagent.

8. A nucleic acid amplification method comprising:
a procedure of dissolving a solid phase reagent at least containing DNA polymerase, cyclodextrin, and a binder, in a liquid containing a nucleic acid; and
a procedure of amplifying the nucleic acid.

9. The nucleic acid amplification method according to claim 8,
wherein amplification of the nucleic acid is isothermally performed.

10. The nucleic acid amplification method according to claim 8,
wherein the nucleic acid is the ribonucleic acid, and
wherein the nucleic acid amplification method further comprises a procedure of performing reverse transcription reaction using a ribonucleic acid as a template prior to the procedure of the amplifying of the nucleic acid.

11. A reagent for solid phase nucleic acid amplification reaction comprising:
at least a DNA polymerase, cyclodextrin, and a binder.

12. The reagent for solid phase nucleic acid amplification reaction according to claim 11,
wherein the cyclodextrin includes a hydroxypropyl group.

13. The reagent for solid phase nucleic acid amplification reaction according to claim 11,
wherein the reagent for solid phase nucleic acid amplification reaction is mixed in a liquid containing a template nucleic acid chain and an ionic surfactant.

14. The reagent for solid phase nucleic acid amplification reaction according to claim 13,
wherein the concentration of the cyclodextrin is higher than or equal to 8 times the concentration of the ionic surfactant.

15. The reagent for solid phase nucleic acid amplification reaction according to claim 13, further comprising ribonuclease H.

16. A microchip comprising a reagent for solid phase nucleic acid amplification reaction at least containing DNA polymerase, cyclodextrin, and a binder.

17. The microchip according to claim 16,
wherein the reagent for solid phase nucleic acid amplification reaction is provided in each of a plurality of reaction sites of nucleic acid amplification reaction installed in the microchip, and
wherein the reaction sites communicate with an entrance through which a liquid enters the microchip, through flow channels.


SEQUENCE LISTING (6 sequences)

SEQ ID NO: 1 - DNA, 19 bp, Artificial Sequence
  The nucleic acid sequence of the primer F3
tgctccggaatagctcctg

SEQ ID NO: 2 - DNA, 18 bp, Artificial Sequence
  The nucleic acid sequence of the primer B3
tgcctcccgtaggagtct

SEQ ID NO: 3 - DNA, 42 bp, Artificial Sequence
  The nucleic acid sequence of the primer FIP
ccaacaagctgataggacgcgacgcatgtgattgtgggaaag

SEQ ID NO: 4 - DNA, 41 bp, Artificial Sequence
  The nucleic acid sequence of the primer BIP
gaggtaacggctcaccaaggcgccgtatctcagtcccaatg

SEQ ID NO: 5 - DNA, 17 bp, Artificial Sequence
  The nucleic acid sequence of the primer LF
ccatcccacgccgatag

SEQ ID NO: 6 - DNA, 19 bp, Artificial Sequence
  The nucleic acide sequence of the QProbe
ccggcctgagagggcgacc